(19) United States
(12) Patent Application Publication
(10) Pub. No.: US 2015/0182629 A1
(43) Pub. Date: Jul. 2, 2015

(54) STABLE COMPOSITIONS OF FESOTERODINE

(71) Applicant: HETERO RESEARCH FOUNDATION, HYDERABAD, ANDHRA PRADESH (IN)

(72) Inventors: Bandi Parthasaradhi Reddy, Hyderabad (IN); Podili Khadgapathi, Hyderabad (IN); Borra Syamprasad, Hyderabad (IN)

(21) Appl. No.: 14/412,339

(22) PCT Filed: Jun. 27, 2013
(86) PCT No.: PCT/IN13/00396

§ 371 Date: Dec. 31, 2014

(30) Foreign Application Priority Data
Jul. 2, 2012 (IN) 2633/CHE/2012

(51) Int. Cl. A61K 47/36; A61K 9/20; A61K 47/14; A61K 47/38; A61K 47/12; A61K 31/195

(52) U.S. Cl. CPC A61K 47/36; A61K 47/12; A61K 31/195; A61K 47/14; A61K 47/38; A61K 9/2059; A61K 9/2054; A61K 9/2013

(57) ABSTRACT

Stable pharmaceutical compositions of fesoterodine or its pharmaceutically acceptable salt thereof and process for preparing the same. In a first embodiment, a stable pharmaceutical composition is provided comprising fesoterodine fumarate, glyceryl behenate and a stabilizer. The stable pharmaceutical tablet composition may further comprise i) fesoterodine fumarate in an 5 amount of 1% to 5% by weight, ii) glyceryl behenate in an amount of 1% to 8% by weight, iii) pregelatinized starch in an amount of 30% to 50% by weight and iv) a stabilizer in an amount of 0.1% to 10% by weight based on total weight of the composition.

### PRIORITY

This patent application claims priority to Indian patent application number 2633/CHE/2012, filed on Jul. 2, 2012, the contents of which are incorporated by reference herein in their entirety.

### FIELD OF THE INVENTION

The present invention encompasses stable pharmaceutical compositions of fesoterodine or pharmaceutically acceptable salts thereof.

### BACKGROUND OF THE INVENTION

Chemically, fesoterodine fumarate is designated as isobutyric acid 2-((R)-3-diisopropylammonium-1phenylpropyl)-4-(hydroxymethyl) phenyl ester hydrogen fumarate. Its empirical formula is C₃₀H₄₁NO₇, corresponding to a molecular weight of 527.66 having the following structural formula:

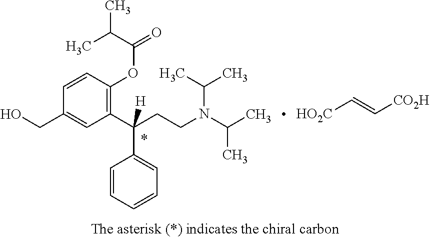 [SMILES: CC(C)C(=O)Oc1ccc(CO)cc1[C@H](CCN(C(C)C)C(C)C)c1ccccc1 ; O=C(O)/C=C/C(=O)O]

Fesoterodine is marketed under the trade name TOVIAZ® in United States by Pfizer in the form of 4 mg and 8 mg tablets for the treatment of overactive bladder with symptoms of urge urinary incontinence, urgency, and frequency.

U.S. Pat. No. 6,858,650 and U.S. Pat. No. 7,384,980 disclose fesoterodine.

U.S. Pat. No. 7,807,715 and U.S. Pat. No. 8,088,398 disclose composition of fesoterodine comprising sorbitol or xylitol, lactose monohydrate, microcrystalline cellulose, hydroxypropyl methylcellulose, glycerol dibehenate and talc.

US20090285891 disclose controlled release composition with at least two diffusion layers and a gastro-resistant coating layer meant for intestinal release.

WO2011117884 claims composition of fesoterodine fumarate with polyvinylpyrrolidone, microcrystalline cellulose and hydroxypropyl methycelllulose.

During storage, fesoterodine compositions yields various impurities in variable quantities, which is not desirable if they are beyond certain limits.

Thus there remains a need to formulate storage stable pharmaceutical compositions of fesoterodine that contain reduced amounts of total impurities.

### SUMMARY OF THE INVENTION

The present invention discloses storage stable pharmaceutical compositions of fesoterodine or a pharmaceutically acceptable salt thereof.

One embodiment of the present invention provides stable pharmaceutical composition comprising fesoterodine fumarate, glyceryl behenate and a stabilizer.

Another embodiment of the present invention provides stable pharmaceutical compositions comprising fesoterodine fumarate in an amount of 1% to 5% by weight, glyceryl behenate in an amount of 1% to 8% by weight, pregelatinized starch in an amount of 30% to 50% by weight, and a stabilizer in an amount of 0.1% to 10% by weight, based on total weight of the composition.

In a specific embodiment, the pharmaceutical tablet compositions of fesoterodine fumarate have reduced levels of 2-((R)-3-(diisopropylamino)-1-phenylpropyl)-4-(hydroxymethyl)phenol impurity when produced and stored for certain period of time.

In another embodiment, the present invention provides a storage stable pharmaceutical composition comprising fesoterodine, having a 2-((R)-3-(di isopropyl amino)-1-phenylpropyl)-4-(hydroxymethyl)phenol impurity in an amount of less than 2% by weight after storage for one month at 40° C. and 75% RH.

In another embodiment, the present invention provides a storage stable controlled release tablet composition comprising fesoterodine fumarate as an active agent; glyceryl behenate as a lubricant; pregelatinized starch as a diluent; hydroxypropyl methylcellulose as controlled-release agent; and a stabilizer selected from citric acid, colloidal silicon dioxide, a mixture of citric acid and pregelatinized starch, hydroxypropyl cellulose, crospovidone, polyethylene glycol and a graft copolymer of polyvinyl alcohol-polyethylene glycol, and combinations thereof.

In another embodiment, the present invention provides a process for the preparation of pharmaceutical tablet composition of fesoterodine comprising: (i) sifting and blending one or more pharmaceutically acceptable excipients with glyceryl behenate to form a dry mixture; (ii) sifting and blending dry mixture of step (i) with fesoterodine; (iii) optionally lubricating the blend of step (ii); and (iv) directly compressing the blend of step (ii) or (iii) into tablets.

In yet another embodiment, the pharmaceutical composition comprising a therapeutically effective amount of fesoterodine is useful for the treatment of overactive bladder with symptoms of urge urinary incontinence, urgency, and frequency.

### DETAILED DESCRIPTION OF THE INVENTION

The present invention discloses storage stable pharmaceutical compositions of fesoterodine or a pharmaceutically acceptable salt thereof.

The term “fesoterodine” as used herein includes fesoterodine in the form of a free base or a pharmaceutically acceptable salt which includes the fumarate, hydrochloride, hydrobromide, nitrate, sulfate, mandelate, oxalate, succinate, maleate, besylatetosylate, palmitate and tartarate salts. More specifically the fesoterodine is fesoterodine fumarate.

The term “storage stable” as used herein refers to solid dosage forms of fesoterodine with reduced levels of total impurities when stored for certain period of time.

Storage stable pharmaceutical compositions of fesoterodine are important to control the levels of impurities in the final dosage form and to ensure that the impurity is present in the lowest possible levels.

The term “composition” or “pharmaceutical composition” or “formulation” or “solid oral composition” or “dosage form” as used herein synonymously include solid dosage forms such as tablets, capsules, granules, mini-tablets and the like meant for oral administration, more specifically tablets.

Compositions of the present invention can be developed into solid oral dosage forms to exhibit extended release, sustained release, controlled release, modified release and delayed release or a combination thereof, using rate controlling polymers. Preferably, the composition of the invention is in the form of an extended release composition.

The term ‘extended release” used herein refer to a composition that provides release of fesoterodine or salts thereof over a period of 24 hours.

The term “effective amount” or “pharmaceutically effective amount” used interchangeably, is defined to mean the amount or quantity of the active drug (e.g., fesoterodine), which is sufficient to elicit an appreciable biological response when administered to the patient. It will be appreciated that the precise therapeutic dose will depend on the age and condition of the patient, nature of the condition to be treated and will be at the ultimate discretion of the attendant physician.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

The term “excipient” means a pharmacologically inactive component such as a diluent, disintegrant, carrier, and the like, of a pharmaceutical product. The excipients that are useful in preparing a pharmaceutical composition are generally safe, non-toxic and are acceptable for veterinary as well as human pharmaceutical use. Reference to an excipient includes both one excipient and more than one excipient.

The present invention provides storage stable pharmaceutical compositions of fesoterodine with one or more pharmaceutically acceptable excipients and process for their preparation.

In one aspect, the present invention provides a stable pharmaceutical composition comprising fesoterodine, having a 2-((R)-3-(diisopropylamino)-1-phenylpropyl)-4-(hydroxymethyl)phenol impurity in an amount of less than 2% by weight after storage for one month at 40° C. and 75% RH, specifically in an amount of less than 1.5% by weight after storage for one month at 40° C. and 75% RH.

In particular, the pharmaceutical tablet compositions of fesoterodine fumarate have reduced levels of 2-((R)-3-(diisopropylamino)-1-phenylpropyl)-4-(hydroxymethyl)phenol impurity.

The present invention also describes selection of suitable pharmaceutically acceptable excipients and/or stabilizers to prepare the stable pharmaceutical compositions of fesoterodine fumarate.

According to the present invention stability of drug-excipient blend was evaluated based on percentage w/w of 2-((R)-3-(diisopropylamino)-1-phenylpropyl)-4-(hydroxymethyl)phenol impurity (H-FTFRC-01) and total impurities present in drug-excipient blend, initially and after storage for one month at 40° C. and 75% relative humidity. The results are presented in Table 1.

TABLE 1
Results of stability evaluation of drug-excipient blend

| | Initial | | 40° C./75% RH, l month | |
|---|---|---|---|---|
| Drug- | H-FTFRC- | Total | H-FTFRC- | Total |
| Excipients | 01* | Impurities | 01* | Impurities |
| Fesoterodine | 0.284 | 0.800 | 0.524 | 1.245 |
| fumarate (FF#) | | | | |
| FF-crospovidone | 0.233 | 0.641 | 0.777 | 1.638 |
| FF-hydroxypropyl | 0.309 | 0.619 | 0.804 | 1.254 |
| cellulose | | | | |
| FF-polyethylene | 0.326 | 0.723 | 1.063 | 1.878 |
| glycol | | | | |
| FF-colloidal silicon | 0.362 | 1.050 | 0.864 | 1.633 |
| dioxide | | | | |
| FF-a graft | 0.425 | 0.838 | 1.011 | 1.8277 |
| copolymer of | | | | |
| polyvinyl alcohol- | | | | |
| polyethylene glycol | | | | |
| FF-a mixture of | 0.370 | 0.610 | 0.590 | 1.050 |
| citric acid and | | | | |
| pregelatinized starch | | | | |
| FF-glyceryl behenate | 0.256 | 0.670 | 0.968 | 1.813 |
| FF-sorbitol | 0.707 | 1.271 | 1.44 | 2.807 |
| FF-lactose | 0.562 | 1.009 | 6.195 | 11.398 |
| monohydrate | | | | |
| FF-microcrystalline | 0.410 | 1.409 | 2.835 | 5.237 |
| cellulose | | | | |
| FF-dibasic calcium | 2.023 | 4.688 | 5.001 | 15.729 |
| phosphate, anhydrous | | | | |
| FF-ethyl cellulose | 1.055 | 1.927 | 3.990 | 5.811 |
| FF-polyethylene | 5.205 | 6.560 | 10.732 | 14.223 |
| oxides | | | | |
| FF-carbopol | 0.183 | 0.747 | 4.053 | 10.438 |
| FF-magnesium | 9.306 | 14.830 | 17.918 | 41.697 |
| hydroxide | | | | |

H-FTFRC-01*: 2-((R)-3-(diisopropylamino)-1-phenylpropy1)-4-(hydroxymethyl) phenol.
FF#: Fesoterodine fumarate.

As can be seen from Table 1, blends of festoterodine fumarate with crospovidone, hydroxypropyl cellulose, polyethylene glycol, a graft copolymer of polyvinyl alcohol-polyethylene glycol, a mixture of citric acid and pregelatinized starch, colloidal silicon dioxide or glyceryl behenate has shown less than about 1 w/w of H-FTFRC-01 impurity after storage for one month at 40° C. and 75% relative humidity. A blend containing lactose monohydrate, microcrystalline cellulose, dibasic calcium phosphate anhydrous, ethyl cellulose, polyethylene oxides, carbopol or magnesium hydroxide, for example, has shown greater than about 1 w/w of H-FTFRC-01 impurity after storage for one month at 40° C. and 75% relative humidity.

Based on the results presented in Table 1, and without being held to theory, it is believed that a mixture of citric acid and pregelatinized starch, colloidal silicon dioxide, crospovidone, hydroxypropyl cellulose, polyethylene glycol, a graft copolymer of polyvinyl alcohol-polyethylene glycol, and glyceryl behenate are compatible with fesoterodine fumarate.

Accordingly, the present invention provides a stable pharmaceutical composition comprising fesoterodine fumarate, glyceryl behenate and a stabilizer.

The present invention also provides a stable pharmaceutical composition comprising fesoterodine fumarate in an amount of 1% to 5% by weight, glyceryl behenate in an amount of 1% to 8% by weight, pregelatinized starch in an amount of 30% to 50% by weight, and a stabilizer in an amount of 0.1% to 10% by weight based on total weight of the composition.

As used herein, a stabilizer is an excipient that protects the fesoterodine fumarate composition from degradation. Exemplary stabilizers include, but are not limited to citric acid monohydrate, colloidal silicon dioxide, a mixture of citric acid and pregelatinized starch, glyceryl behenate, hydroxypropyl cellulose, crospovidone, polyethylene glycol and a graft copolymer of polyvinyl alcohol-polyethylene glycol or a combination thereof.

In certain embodiments, pharmaceutical compositions of the present invention further comprise a controlled release agent.

Controlled-release agents provide gradual release of fesoterodine over an extended period of time. Exemplary controlled-release agents include, but are not limited to hydroxypropyl methylcellulose, hydroxypropyl cellulose, polyacrylates, methyl acrylates, polyethylene glycols, chitosan, gums, starch derivatives, polyurethanes, galactomannans, polysaccharides, polyalcohols, glycerol palmitostearate, beeswax, glycowax, carnaubawax, hydrogenated vegetable oil, glycerol monostearate, stearyl alcohol, polyanhydrides, methyl acrylates, and the like, and combinations thereof. A specific controlled-release agent is hydroxypropyl methylcellulose. More specifically hydroxypropyl methylcellulose having a viscosity of 100 cps to 1,00,000 cps is used.

In one embodiment, a controlled-release fesoterodine formulation releases fesoterodine over a period of 24 hours.

In one embodiment, a pharmaceutical composition of the present invention is free from sugar alcohols. The term “sugar alcohols” used herein refers to polyhydric sugars and its derivatives, like mannitol, xylitol, maltitol, isomaltitol, erythritol, lactitol and sorbitol. “Free from sugar alcohols” means that the composition has no added sugar alcohols.

The compositions of fesoterodine optionally further comprise one or more pharmaceutically acceptable excipients selected from a diluent, a disintegrant, a glidant and a lubricant or a combination thereof.

Exemplary diluents include pregelatinized starch, starches, modified starches, and the like, and a combinations thereof.

Exemplary disintegrants include crospovidone, croscarmellose sodium, sodium starch glycolate, polacrillin potassium, polyvinylpyrrolidone, starches such as corn starch, potato starch, pre-gelatinized and modified starches, clays, bentonite, and the like, and combinations thereof.

Exemplary glidants include talc, colloidal silicon dioxide and other forms of silicon dioxide, and combinations thereof.

Suitable lubricants include glyceryl behenate, talc, calcium stearate, sodium stearyl fumarate, zinc stearate, stearic acid, fumaric acid, palmitic acid, and the like, and combinations thereof.

The compositions optionally include a film coat. A film coat on the tablet provides an elegant appearance, protects from moisture and further contributes to the ease with which it can be swallowed.

In one embodiment, it has further been observed by the present inventors that, fesoterodine fumarate compositions prepared by direct compression process were more stable as compared to compositions prepared by dry granulation process and/or wet granulation process.

The present invention provides a process for the preparation of pharmaceutical composition comprising fesoterodine and one or more pharmaceutically acceptable excipients, using direct compression.

In first aspect, a direct compression process comprises:

(i) sifting and blending one or more pharmaceutically acceptable excipients with glyceryl behenate to form a dry mixture;

(ii) sifting and blending dry mixture of step (i) with fesoterodine;

(iii) optionally lubricating the blend of step (ii); and

(iv) directly compressing the blend of step (ii) or (iii) into tablets.

In second aspect, a direct compression process comprises:

(i) sifting and blending one or more excipients with a first portion of glyceryl behenate to form a dry mixture;

(ii) sifting and blending a portion of dry mixture of step (i) with fesoterodine fumarate;

(iii) blending the materials of step (i) and (ii);

(iv) lubricating the blend of step (iii) with a second portion of glyceryl behenate to form a lubricated blend; and

(v) directly compressing the lubricated blend of step (iv) into tablets.

In one embodiment of the method of forming a composition, the first and second portions of glyceryl benenate in steps (i) and (iv) are each half of the total amount of glyceryl behenate in the composition. In another embodiment, the portion of dry mixture of step (i) employed in step (ii) is 5-20%, specifically 10% of the dry mixture of step (i).

In a specific aspect, the process for preparing stable pharmaceutical compositions of fesoterodine involves pre-lubrication of excipients with glyceryl behenate to reduce the interaction between drug and excipients. This pre-lubrication results significantly reduced level of 2-((R)-3-(diisopropylamino)-1-phenylpropyl)-4-(hydroxymethyl)phenol impurity and total impurities in the final dosage form as compared to compositions prepared by a conventional blending process. The stability of dosage forms of fesoterodine fumarate can be improved based on sequence of mixing drug and excipients during process.

The pharmaceutical composition comprising therapeutically effective amount of fesoterodine or its pharmaceutically acceptable salt thereof is useful for the treatment of overactive bladder with symptoms of urge urinary incontinence, urgency, and frequency.

Certain specific aspects and embodiments of this invention are described in further detail by the examples below, which are provided only for purposes of illustration and are not intended to limit the scope of the invention in any manner.

### Example-1

### Pre-Lubrication of Excipients with Glyceryl Behenate

### Fesoterodine Tablets Prepared According to the Composition Listed in Table 2 Using the Following Steps:

1. Pregelatinized starch, citric acid monohydrate, hydroxypropyl methylcellulose K100 CR, hydroxypropyl methylcellulose K100M CR, hydroxypropyl methylcellulose K4M, and colloidal silicon dioxide were sifted with a half quantity of glyceryl behenate through a mesh #40 sieve and dry blended for 15 minutes.
2. 10% blend of step 1 and fesoterodine hydrogen fumarate were sifted together through a mesh #40 sieve.
3. sifted materials of step 1 and step 2 were blended for 10 minutes.
4. remaining half quantity of glyceryl behenate was sifted through a mesh #60 sieve.
5. blend of step 3 was lubricated with glyceryl behenate of step 4.
6. lubricated blend of step 5 was directly compressed into tablets.
7. tablets of step 6 were film coated using an Opadry® dispersion.

TABLE 2
Fesoterodine tablet compositions prepared
by direct compression process:

| Ingredient | mg/Tablet |
|---|---|
| Fesoterodine hydrogen fumarate | 8.00 |
| Pregelatinized starch | 147.44 |
| Hydroxypropyl methylcellulose K 100 CR | 72.00 |
| Hydroxypropyl methylcellulose K 100M CR | 48.00 |
| Hydroxypropyl methylcellulose K4M CR | 24.00 |
| Citric acid monohydrate | 0.66 |
| Colloidal silicon dioxide | 9.90 |
| Glyceryl behenate | 10.00 |
| Core tablet weight | 320.00 |
| Film coating | |
| Opadry ® blue | 10.00 |
| Coated tablet weight | 330.00 |

Dissolution study:

| | |
|---|---|
| Dissolution Medium | 6.8 pH phosphate buffer |
| Volume | 900 ml |
| Apparatus | USP II (Paddle) |
| Speed | 75 RPM |

| Time in Hours | 1 | 2 | 4 | 6 | 8 | 12 | 16 | 20 |
|---|---|---|---|---|---|---|---|---|
| Example-1 | 16 | 25 | 38 | 48 | 58 | 73 | 83 | 89 |
| Dissolution (%) | | | | | | | | |

### Comparative Example-2

### Normal Blending Process

### Fesoterodine Tablet Composition as Described in Example-1 was Repeated, Using the Following Steps:

1. Fesoterodine hydrogen fumarate, pregelatinized starch, citric acid monohydrate, hydroxypropyl methylcellulose K100 CR, hydroxypropyl methylcellulose K100M CR, hydroxypropyl methylcellulose K4M and colloidal silicon dioxide were sifted through a mesh #40 sieve and blended for 15 minutes.
2. glyceryl behenate was sifted through a mesh #60 sieve.
3. blend of step 1 was lubricated with the glyceryl behenate of step 2.
4. lubricated blend of step 3 was directly compressed into tablets.
5. tablets of step 4 were film coated using an Opadry® dispersion.

The film coated tablets obtained in Example 1 and Comparative Example 2 were evaluated for stability over a period of 1 month at 40° C. and 75% relative humidity for the presence of impurities using HPLC, initially and after storage for one month. The results of this measurement were listed as percentage w/w of 2-((R)-3-(diisopropylamino)-1-phenylpropyl)-4-(hydroxymethyl)phenol impurity and total impurities in Table 3.

TABLE 3
Results of stability evaluation of fesoterodine film coated tablets

| | Initial | | 40° C./75% RH, l month | |
|---|---|---|---|---|
| | H-FTFRC- | Total | H-FTFRC- | Total |
| Blending process | 01* | Impurities | 01* | Impurities |
| Example-1 | 0.319 | 0.648 | 1.111 | 1.424 |
| (Pre-lubrication of | | | | |
| excipients with | | | | |
| glyceryl behenate) | | | | |
| Comparative | 0.547 | 0.902 | 1.789 | 2.566 |
| Example-2 | | | | |
| (Normal blending | | | | |
| process) | | | | |

H-FTFRC-01*: 2-((R) -3- (diisopropylamino)-1-phenylpropy1)-4-(hydroxymethyl)phenol.

As can be seen from Table 3, Inventive Example-1 has reduced levels of H-FTFRC-01 impurity and total impurities as compared to Comparative Example-2, initially and after storage for one month at 40° C. and 75% relative humidity.

Based on the results presented in Table 3, there appears to be a correlation between the formation of impurities and order of mixing drug and excipients in blending process. The formulation of Example-1 involves pre-lubrication of excipients which reduces the interaction between drug and excipients. Initially and after storage for one month, Example-1 had significantly lower levels of 2-((R)-3-(diisopropylamino)-1-phenylpropyl)-4-(hydroxymethyl)phenol impurity and total impurities. In contrast, the formulation of Comparative Example-2 prepared by a conventional blending process, had significantly higher level of 2-((R)-3-(diisopropylamino)-1-phenylpropyl)-4-(hydroxymethyl)phenol impurity and total impurities initially and after storage when compared to formulation Example-1. Thus, the data in Table 3 indicates that the stability of dosage forms of fesoterodine fumarate can be improved based on order/sequence of mixing drug and excipients.

### Example 3-5

TABLE 4
Fesoterodine tablet compositions prepared by granulation process:

| | Example-3 | Example-4 | Example-5 |
|---|---|---|---|
| | mg/Tablet | mg/Tablet | mg/Tablet |
| | (Dry | (Wet | (Wet |
| Ingredients | granulation) | granulation) | granulation) |
| Intra-granular ingredients | | | |
| Fesoterodine fumarate | 8.00 | 8.00 | 8.00 |
| Pregelatinized starch | 147.44 | 147.44 | 147.44 |
| Hydroxypropyl | 72.00 | 72.00 | 72.00 |
| methylcellulose | | | |
| K100 CR | | | |
| Hydroxypropyl | 48.00 | 48.00 | 48.00 |
| methylcellulose | | | |
| K100M CR | | | |
| Hydroxypropyl | 24.00 | 24.00 | 24.00 |
| methylcellulose | | | |
| K4M | | | |
| Citric acid | 0.66 | 0.66 | 0.66 |
| monohydrate | | | |
| Colloidal | 9.90 | 9.90 | 9.90 |
| silicon dioxide | | | |
| Glyceryl behenate | 5.00 | 5.00 | 5.00 |
| Granulation | | | |
| Purified water | — | q.s. | — |
| Isopropyl alcohol | — | — | q.s. |
| Extragranular | | | |
| ingredients | | | |
| Glyceryl behenate | 5.00 | 5.00 | 5.00 |
| Core tablet weight | 320.0 | 320.0 | 320.0 |
| Film coating | | | |
| Opadry ® blue | 10.00 | 10.00 | 10.00 |
| Film coated tablet weight | 330.00 | 330.00 | 330.00 |

### Manufacturing Process for Example 3-5:

1. Pregelatinized starch, citric acid monohydrate, hydroxypropyl methylcellulose K100 CR, hydroxypropyl methylcellulose K100M CR, hydroxypropyl methylcellulose K4M and colloidal silicon dioxide were sifted with a half quantity of glyceryl behenate through a mesh #40 sieve and blended for 15 minutes.
2. 10% of the blend of step 1 and fesoterodine fumarate were sifted together through a mesh #40 sieve.
3. sifted materials of step 1 and 2 were blended for 10 minutes.
4. blend of step 3 was slugged/compacted or wet granulated using purified water or isopropyl alcohol, followed by milling and sifting to provide the desired granules.
5. remaining half quantity of glyceryl behenate was sifted through a mesh #60 sieve.
6. granules of step 4 were lubricated with glyceryl behenate of step 5.
7. lubricated blend of step 6 was compressed into tablets.
8. tablets of step 7 were film coated using an Opadry® dispersion.

The tablets obtained in Examples 3 to 5 were subjected to a stability evaluation at 40° C. and 75% relative humidity. The tablets were evaluated for the presence of 2-((R)-3-(diisopropylamino)-1-phenylpropyl)-4-(hydroxymethyl)phenol impurity as well as total impurities using HPLC. The results are shown in Table 5.

TABLE 5
Results of stability evaluation of fesoterodine tablets

| | H-FTFRC-01* | Total |
|---|---|---|
| Process | impurity | impurities |
| Example-2 | 0.319 | 0.648 |
| Direct compression | | |
| Example-3 | 0.66 | 1.02 |
| Dry granulation | | |
| Example-4 | | |
| Wet granulation | 1.470 | 1.839 |
| (with aqueous solvent) | | |
| Example-5 | 6.119 | 7.229 |
| Wet granulation | | |
| (with isopropyl alcohol) | | |

H-FTFRC -01* : 2-((R) -3- (diisopropylamino)-1-phenylpropy1)-4-(hydroxymethyl) phenol.

As can be seen from Table 5, Example-2 has reduced amounts of 2-((R)-3-(diisopropylamino)-1-phenylpropyl)-4-(hydroxymethyl)phenol impurity and total impurities at 40° C. and 75% relative humidity, as compared to Examples 3, 4 and 5.

Based on the results presented in Table 5, there appears to be a correlation between the formation of impurities and process for the preparation of composition in addition to the excipients used. The formulation of Example-2 prepared by a direct compression process had significantly lower levels of impurities relative to the formulation of Example 3 (dry granulation process) and Examples 4 and 5 (wet granulation process using purified water and isopropyl alcohol respectively). Thus, the data in Table 5 indicates that fesoterodine fumarate compositions prepared by direct compression process were more stable as compared to compositions with dry granulation process and/or wet granulation process.

### Example 6

### Fesoterodine Tablet Compositions Prepared by Direct Compression Process

| Ingredient | mg/Tablet |
|---|---|
| Fesoterodine fumarate | 8.00 |
| Pregelatinized starch | 116.10 |
| Hydroxypropyl | 72.00 |
| methylcellulose K 100 CR | |
| Hydroxypropyl | 48.00 |
| methylcellulose K 100M CR | |
| Hydroxypropyl | 24.00 |
| methylcellulose K 4M CR | |
| Hydroxypropyl cellulose | 32.00 |
| Colloidal silicon dioxide | 9.90 |
| Glyceryl behenate | 10.00 |
| Core tablet weight | 320.00 |
| Film coating | |
| Opadry ® blue | 10.00 |
| Coated tablet weight | 330.00 |

### Manufacturing Process:

1. Pregelatinized starch, hydroxypropyl methylcellulose K 100 CR, hydroxypropyl methylcellulose K 100M CR, hydroxypropyl methylcellulose K4M CR, hydroxypropyl cellulose, colloidal silicon dioxide were sifted with a half quantity of glyceryl behenate through a mesh #40 sieve and blended for 15 minutes.
2. 10% of the blend of step 1 and fesoterodine fumarate were sifted together through a mesh #40 sieve.
3. sifted materials of step 1 and 2 were blended for 10 minutes.
4. remaining half quantity of glyceryl behenate was sifted through a mesh #60 sieve.
5. blend of step 3 was lubricated with the glyceryl behenate of step 4.
6. lubricated blend of step 5 was directly compressed into tablets.
7. tablets of step 6 were film coated using an Opadry® dispersion.

### Example 7

### Fesoterodine Tablet Compositions Prepared by Direct Compression Process

| Ingredient | mg/Tablet |
|---|---|
| Fesoterodine fumarate | 8.00 |
| Hydroxypropyl | 72.00 |
| methylcellulose K 100 CR | |
| Hydroxypropyl | 48.00 |
| methylcellulose K 100M CR | |
| Hydroxypropyl | |
| methylcellulose K4M CR | 24.00 |
| Crospovidone | 149.50 |
| Colloidal silicon dioxide | 8.50 |
| Glyceryl behenate | 10.00 |
| Core tablet weight | 320.00 |
| Film coating | |
| Opadry ® blue | 10.00 |
| Coated tablet weight | 330.00 |

### Manufacturing Process:

1. Hydroxypropyl methylcellulose K 100 CR, hydroxypropyl methylcellulose K 100M CR, hydroxypropyl methylcellulose K4M CR, crospovidone, colloidal silicon dioxide and half quantity of glyceryl behenate were sifted together through mesh #40 and blended for 15 min.
2. 10% blend of step 1 and fesoterodine fumarate were sifted together through mesh #40.
3. sifted materials of step 1 and 2 were blended for 10 minutes.
4. remaining half quantity of glyceryl behenate was sifted through mesh #60.
5. blend of step 3 was lubricated with glyceryl behenate of step 4.
6. lubricated blend of step 5 was compressed into tablets.
7. tablets of step 6 were film coated using an Opadry® dispersion.

1. A stable pharmaceutical composition comprising fesoterodine fumarate, glyceryl behenate and a stabilizer,
wherein the stabilizer is citric acid, or a combination of citric acid and pregelatinized starch.

2. A stable pharmaceutical tablet composition comprising i) fesoterodine fumarate in an amount of 1% to 5% by weight, glyceryl behenate in an amount of 1% to 8% by weight, iii) pregelatinized starch in an amount of 30% to 50% by weight and iv) a stabilizer in an amount of 0.1% to 10% by weight based on total weight of the composition.

3. The stable pharmaceutical composition of claim 1, wherein the stabilizer is citric acid.

4. The stable pharmaceutical composition of claim 2, wherein the composition is an extended-release composition.

5. The stable pharmaceutical composition of claim 4, further comprising hydroxypropyl methylcellulose as a controlled-release agent, wherein the hydroxypropyl methylcellulose has a viscosity of 100 cps to 1,00,000 cps.

6. The stable pharmaceutical composition of claim 1, wherein the composition has a 2-((R)-3-(diisopropylamino)-1-phenylpropyl)-4-(hydroxymethyl)phenol impurity in an amount of less than 2% by weight after storage for one month at 40° C. and 75% RH.

7. The stable pharmaceutical composition of claim 1, further comprising a diluent, a binder, a disintegrant, a lubricant, a glidant, or a combination thereof.

8. The stable pharmaceutical tablet composition of claim 2, further comprising a film coating.

9. The stable pharmaceutical tablet composition of claim 2, wherein the tablet is prepared by direct compression.

10. A process for the preparation of pharmaceutical tablet composition of fesoterodine comprising:
(i) sifting and blending one or more pharmaceutically acceptable excipients with glyceryl behenate to form a dry mixture;
(ii) sifting and blending dry mixture of step (i) with fesoterodine;
(iii) optionally lubricating the blend of step (ii); and
(iv) directly compressing the blend of step (ii) or (iii) into tablets.

11. The process of claim 10, wherein the pharmaceutical composition is that of claim 1.

12. The stable pharmaceutical composition of claim 1, further comprising pregelatinized starch as a diluent; hydroxypropyl methylcellulose as controlled-release agent; wherein the stable pharmaceutical composition is in the form of a controlled release tablet.

13. The stable pharmaceutical composition according to claim 1 wherein the composition is free from added sugar alcohols.

14. A method of treating overactive bladder with symptoms of urge urinary incontinence, urgency, and frequency in a patient in need thereof, comprising administering to the patient the composition of claim 2.

15. The stable pharmaceutical tablet composition according to claim 2 wherein the composition is free from added sugar alcohols.

16. The stable pharmaceutical tablet composition of claim 2, wherein the stabilizer is citric acid, or a combination of citric acid and pregelatinized starch.

17. The stable pharmaceutical tablet composition of claim 2, wherein the stabilizer is citric acid

18. The stable pharmaceutical composition of claim 2, wherein the composition has a 2-((R)-3-(diisopropylamino)-1-phenylpropyl)-4-(hydroxymethyl)phenol impurity in an amount of less than 2% by weight after storage for one month at 40° C. and 75% RH.

19. The stable pharmaceutical composition of claim 2, further comprising a diluent, a binder, a disintegrant, a lubricant, a glidant, or a combination thereof.

20. The process of claim 10, wherein the pharmaceutical composition is that of claim 2.
